(19) United States
(12) Patent Application Publication
(10) Pub. No.: US 2006/0068384 A1
(43) Pub. Date: Mar. 30, 2006

(54) Immuno polymerase chain reaction assay

(75) Inventors: David Thomas McCreavy, Liverpool (GB); William Duncan Fraser, Liverpool (GB); James Anthony Gallagher, Liverpool (GB)

Correspondence Address: CROWELL & MORING LLP;INTELLECTUAL PROPERTY GROUP, P.O. BOX 14300, WASHINGTON, DC, 20044-4300, US

(73) Assignee: UNIVERSITY OF LIVERPOOL Senate House, Liverpool (GB)

(21) Appl. No.: 10/497,778

(22) PCT Filed: Dec. 4, 2002
(86) PCT No.: PCT/GB02/05485

§ 371 Date: Jul. 27, 2005

(30) Foreign Application Priority Data
Dec. 7, 2001 (GB) 0129312.5
Aug. 13, 2002 (GB) 0218733.4

(51) Int. Cl. C12Q 1/68

(52) U.S. Cl. 435/6

(57) ABSTRACT

A method to detect biological molecules in a sample which is an immuno polymerase chain reaction assay which comprises the detection of a complex which includes a ligand: nucleic acid conjugate bound to at least one biological molecule. The complex is detected by the addition of a second nucleic acid molecule which is adapted to anneal to the nucleic acid of the conjugate.

The invention relates to an immunoassay which utilises the polymerase chain reaction.

The use of antibodies as a diagnostic tool is well documented, see Diagnostic Applications of Monoclonal Antibodies to Human Cancer, Arch Pathol Lab Med 108, 2, 101-105 (1985); Monoclonal Antibodies and Colorectal Carcimoma: a clinical Review of Diagnostic Applications 12, 3, 314-323 (1994); Diagnostic Approach to Phospholipid-Dependent Antibodies: State of the Art Lecture Haemostasis, 29, 2-3, 135-149. Monoclonal antibodies, in particular, are advantageous since they are highly specific for epitopes found in antigens which means a high degree of specificity can be obtained when testing for antigens which are specifically expressed in disease tissue providing a reliable measure of the expression of the antigen and accurate diagnosis of a disease which is correlated with expression of the antigen (i.e. tumour rejection antigens in cancer). It is also well documented that the detection of nucleic acid by, for example hybridisation or the polymerase chain reaction (PCR), using nucleic acid probes has also been used in the diagnosis of disease. DNA probes can be relatively easily and inexpensively synthesized using oligonucleotide synthesis. The specificity of a nucleic acid probe is determined by its sequence and how homologous the sequence is to the nucleic acid which is to be detected. However a problem associated with both antibodies and nucleic acid probes is a degree of non-specific binding of the antibody or nucleic acid probe to assay products and/or other proteins and nucleic acid. A diagnostic test has to have a very high degree of reliability if it is to have value in predicting the early on set of disease.

There are a number of molecules present within serum, for example, interleukins and parathyroid hormone related protein, which are potential markers of cancer and other pathological conditions. Currently these are only measurable during the late stages of the disease process when they are overexpressed by tumours. Under normal conditions the proteins are present at concentrations <0.1 pM. Moreover, the early detection of pathogenic organisms in an infection can be critical to whether or not an infected animal survives the infection This is particularly the case in diseases such as bacterial meningitis and septicemia caused, for example by Staphyloccocus aureaus. The earlier these molecules can be measured during the disease process the better the prognosis. However, early detection means that the molecules are at low concentrations and the signaling/quantitation systems of current immunoassays, using enzymes and chemiluminescence does not provide sufficient sensitivity to measure at these low levels.

In addition, there are many clinical scenarios where the simultaneous measurement of more than one analyte in a sample would be of significant diagnostic and therapeutic benefit. Some obvious examples relate to the investigation of endocrine abnormalities where the interpretation of a single result would be significantly affected by another result.

In thyroid disease a combination of thyroid stimulating hormone (TSH) with an index of thyroid hormone status (total or free thyroid hormone) is essential to make an accurate diagnosis in many cases. In investigation of calcium disorders a combination of Parathyroid Hormone (PTH) and Parathyroid Hormone Related Protein (PTHrP) would differentiate the two major causes of hypercalcaemia and elucidate the presence of dual pathology which is often overlooked. When problems of sexual dysfunction and infertility are investigated the pituitary hormones luteinising hormone (LH) and follicle stimulating hormone (FSH) are measured with oestradiol and/or testosterone included in the hormone profile. Another major diagnostic area is the screening for Down's syndrome where a combination of hormone measurements (Human Chorionic Gonadotrophin (HCG), alpha feto-protein (αFP)) and maternal age is used to assess risk of the fetus having Down's syndrome. It is clear that the development of an assay system able to simultaneously measure a number of analytes would have numerous diagnostic applications.

Sano et al (Science (1992), 258:120-122) has developed a technique in which a biotinylated double stranded DNA (dsDNA) template is bound to a biotinylated detector antibody during the final stages of an immunoassay using streptavidin. PCR is undertaken to amplify the dsDNA, quantitation being achieved through gel electrophoresis and scanning equipment. The technique is known as “immuno-PCR”

Immuno-PCR is a method which combines both antibody technology and the polymerase chain reaction or other means to detect a nucleic acid probe conjugated to the antibody. In essence, immuno-PCR utilises an antibody to which a nucleic acid probe has been conjugated. The conjugate binds an antigen to be detected via the antibody part and non-bound conjugate is washed from the sample. The bound antibody is then detected by a PCR reaction which amplifies the nucleic acid part of the conjugate. The assay provides a sensitive and specific test for a biological molecule which benefits from both the advantages discussed above. Specificity is provided by the antibody and sensistivity by the PCR detection of the nucleic acid conjugated to the antibody.

In WO94/26932 an immuno-PCR method is disclosed which is referred to as Nucleic Acid Tagged Immunoassay or NATIA which involves the immobilisation of an antigen or antibody to a solid support. In this example either the antibody is conjugated to an oligonucleotide which is then use to detect the immobilised antigen or the antibody is immobilised and the antigen conjugated to the oligonucleotide. In either case the bound antibody/antigen is detected indirectly by PCR amplification.

In WO9632640 a variation on immuno-PCR is disclosed. The assay described utilises an RNA dependent RNA polymerase. The conjugate comprises an antibody and a DNA template for an RNA transcript. The bound conjugate is detected by transcribing the DNA template into RNA using an RNA dependent RNA polymerase, such as QB replicase. The enzyme is able to transcribe RNA from the DNA template but with lower efficiency than a RNA template. Methods to conjugate nucleic acid to protein molecules are known in the art. For example U.S. Pat. No. 5,635,602 discloses antibody/DNA conjugates and methods for making same.

These techniques demonstrate increased sensitivity. However there are disadvantages associated with the prior art techniques. For example, background resulting from non-specific binding via the nucleic acid moiety or the antibody to plastic of the assay wells is problematic. Moreover the quantitation sytems are insensitive. In order to address these issues we have developed an alternative approach termed Multiple Analyte Quantitation through Single Stranded Extension (MAQSSE).

An oligonucleotide is conjugated to a ligand which has specificity for a biological molecule. The ligand/DNA conjugate is incubated with a sample and binds a target biological molecule. A single stranded DNA template (ssDNA) of defined length is then added to the reaction and anneals to the bound oligonucleotide. A DNA polymerase and deoxynucleotide triphosphates are added and the reaction heated to to elongate the primed oligonucleotide strand to produce a dsDNA. A nuclease specific for ssDNA is added to the reaction to degrade the background ssDNA template, resulting in no detectable background template. To generate a measureable signal, PCR is undertaken to amplify the double stranded DNA followed by detection using conventional techniques.

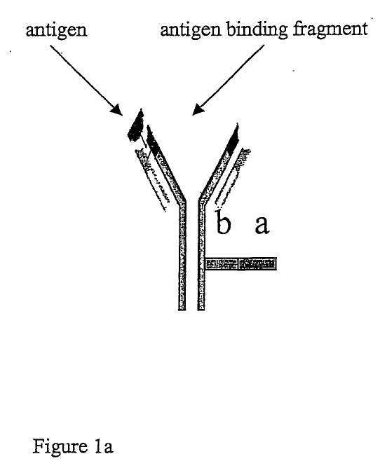

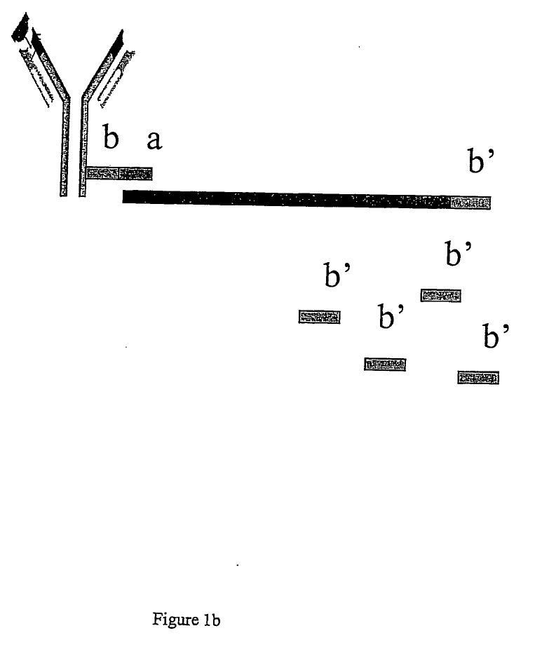

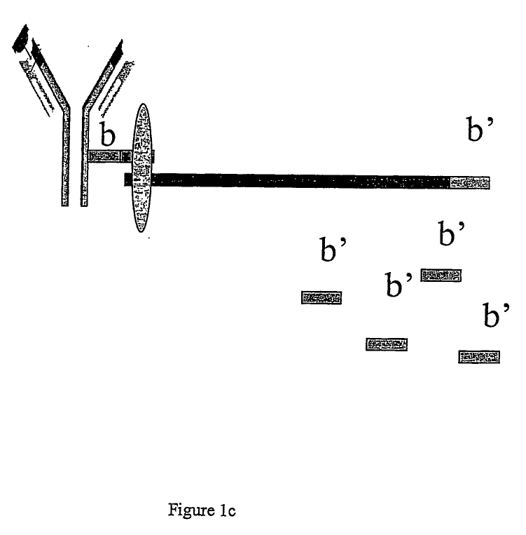

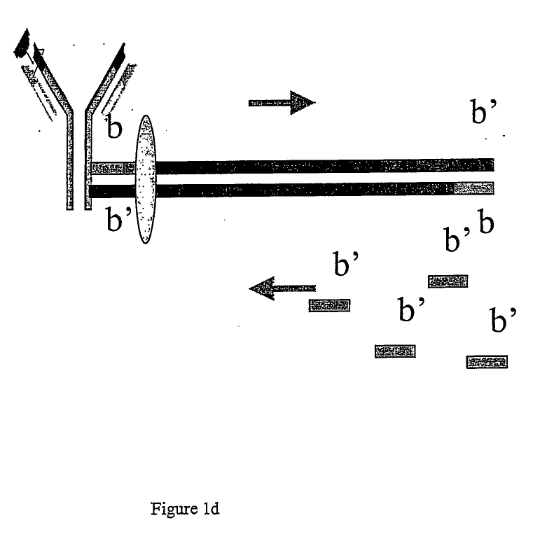

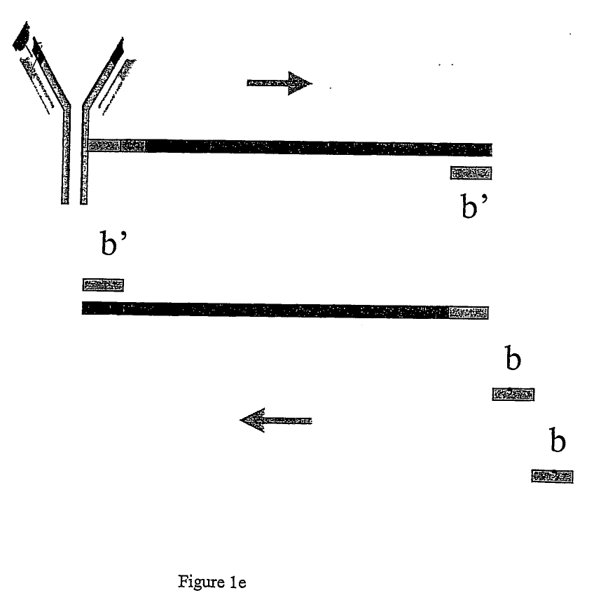

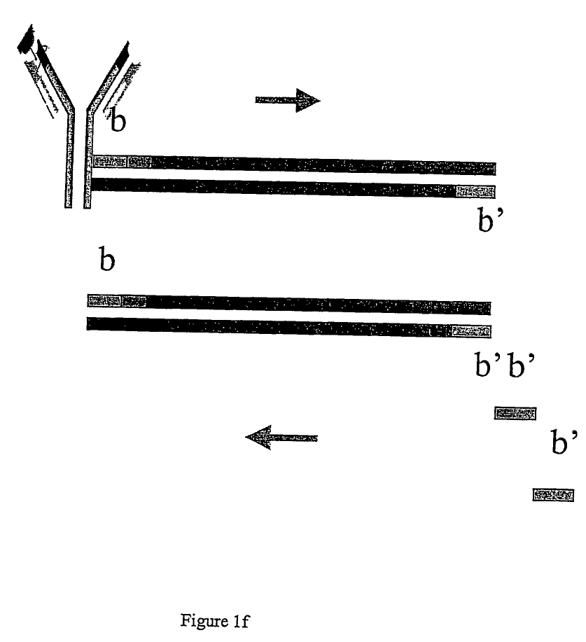

A variation of the above method is also disclosed which greatly simplifies the assay and removes the need to add an exogenous single stranded nuclease to remove the ssDNA template remaining in the reaction mix. The variation comprises a ligand:oligonucleotide conjugate wherein the oligonucleotide has a bipartite sequence structure, (illustrated in FIG. 1 as “a,” and “b”). The conjugate thus formed is contacted with a test sample which potentially includes biological molecule to which the ligand binds.

The bound conjugate is then incubated with the ssDNA template. The bipartite oligonucleotide is complementary over part of its length to a region of the ssDNA. The annealed bipartite oligonucleotide is extended by DNA polymerase to form a double standed DNA. An excess of oligonucleotide primer is added to the reaction mix, the sequence of which is complementary to that part of the bipartite oligonucleotide which is not annealed to the ssDNA template. A polymerase chain reaction is then conducted. Only the ssDNA which has annealed to the bipartite oligonucleotide is capable of being subsequently amplified therefore the assay provides a highly specific and sensitive means to monitor the presence of biological molecules.

According to an aspect of the invention there is provided a method to detect at least one biological molecule comprising providing means to detect said biological molecule which means comprises a ligand:nucleic acid conjugate wherein the binding of the conjugate to said biological molecule is detected by a polymerase chain reaction which detects a second nucleic acid molecule which is adapted to anneal to the nucleic acid of the conjugate.

According to a further aspect of the invention there is provided a method to detect at least one biological molecule wherein said method comprises the steps of:

i) providing a preparation comprising;
a) an assay sample to be tested; and
b) a ligand which is coupled to an oligonucleotide wherein said ligand can bind at least one biological molecule in said sample;

ii) incubating said preparation under conditions which allow the binding of said ligand to said biological molecule to form a complex;

iii) contacting the complex formed in (ii) with a single stranded nucleic acid molecule adapted to anneal to at least part of the oligonucleotide coupled to said ligand;

iv) providing a polymerase which is capable of elongating the oligonucleotide annealed to said single stranded nucleic acid molecule to form a double stranded nucleic acid;

v) incubating the preparation formed in (iii) with a nuclease which degrades the unannealed single stranded nucleic acid molecule;

vi) providing a polymerase and reaction conditions which amplify the double stranded nucleic acid molecule in (iv); and optionally

vii) detecting the presence of the amplified product formed in (vi).

In a preferred method of the invention there is provided an assay sample selected from the group consisting of a sample of: blood; serum; semen; lymph fluid; cerebrospinal fluid; tears; saliva; urine; sweat.

In a further preferred method of the invention said ligand is a polypeptide.

In a yet further preferred method of the invention said polypeptide is an antibody, or at least the effective binding part thereof. Preferably said antibody is a monoclonal antibody, or at least the Fab fragment of said monoclonal antibody.

In a further preferred method of the invention said biological molecule is associated with a disease condition, for example cancer e.g. a tumour rejection antigen. Tumour rejection antigens are known in the art, for example and not by way of limitation, the MAGE, BAGE, GAGE and DAGE families of tumour rejection antigens, see Schulz et al Proc Natl Acad Sci USA, 1991, 88, pp 991-993. Other examples include hormones e.g. thyroid stimulating hormone.

In a further preferred method of the invention said biological molecule is a polypeptide, preferably an antigenic polypeptide expressed by a pathogen. For example a viral, bacterial or parasitic pathogen.

In a further preferred method of the invention said polypeptide is a receptor. Alternatively said polypeptide is a ligand for a receptor.

It will be clear to someone skilled in the art that the ligand can be an antibody which is specific for a biological molecule which may be present in said assay sample. Alternatively the biological molecule may be labelled with the oligonucleotide and the antibody specific for said biological molecule detected in the assay sample.

In a further preferred method of the invention said polymerase is a DNA polymerase. Preferably said DNA polymerase is selected from the group consisting of: E.coli DNA polymerase I; large fragment of E.coli DNA polymerase I, also referred to as Klenow fragment; T4 and T7 bacteriophage DNA polymerase; modified T7 bacteriophage polymerase (referred to as Sequenase™).

Thermostable DNA polymerases are also included although these enzymes only have optimal activity at 70-80° C. Thermostable DNA polymerases will have reduced activity at the reaction temperatures used in the method according to the invention but they nevertheless have activity. Examples of thermostable DNA polymerases are so called Taq polymerase isolated from the thermophilic bacterium, Thermus aquaticus. Other examples include thermostable DNA polymerases isolated from Thermus thermophilus; Thermosipho africanus; Thermotosa maritima.

In a preferred embodiment of the invention said DNA polymerase is T4 bacteriophage DNA polymerase.

In a further preferred embodiment of the invention said nuclease is a single stranded nuclease. Preferably said single stranded nuclease is S1 nuclease or mung bean nuclease.

In a further preferred embodiment of the invention the polymerase used to amplify said double stranded nucleic acid is a thermostable DNA polymerase. Preferably a thermostable DNA polymerase as hereinbefore described.

The amplified product may be analysed by means known in the art which allow the detection and/or quantitation of the DNA product. Typically this includes spectroscopy; fluorimetry, gel electrophoresis (agarose, polyacrylamide).

According to a further aspect of the invention there is provided a ligand:oligonucleotide conjugate wherein said oligonucleotide is adapted, over at least part of its length, to anneal to a single stranded nucleic acid by complementary base pairing.

According to a further aspect of the invention there is provided a conjugate according to any previous aspect of the invention which further comprises an annealed single stranded nucleic acid molecule.

According to a yet further aspect of the invention there is provided a conjugate according to any previous aspect of the invention which comprises an annealed single stranded nucleic acid molecule wherein said conjugate is bound to the ligand binding domain of at least one biological molecule.

In a preferred embodiment of the invention said oligonucleotide is at least 10 base pairs long. Preferably, at least 20 base pairs long. More preferably still said oligonucleotide is between 10-50 base pairs long.

Preferably said second nucleic acid is a single stranded DNA.

In a further preferred embodiment of the invention said ligand is a polypeptide. Preferably said ligand is an antibody, or the effective binding part thereof. Preferably said antibody is a monoclonal antibody.

According to a further aspect of the invention there is provided a method to detect at least one biological molecule comprising the steps of:

i) providing a preparation comprising;
a) an assay sample to be tested; and
b) a conjugate according to the invention;

ii) incubating said preparation under conditions which allow the binding of said conjugate to said biological molecule to form a complex;

iii) contacting the complex formed in (ii) with a single stranded nucleic acid molecule adapted to anneal to a part of the oligonucleotide in said conjugate;

iv) providing a polymerase which is capable of elongating and amplifying the annealed oligonucleotide and at least one oligonucleotide primer wherein said primer comprises a sequence complementary to that part of the oligonucleotide to which said single stranded nucleic acid is not annealed; and optionally

v) detecting the presence of the amplified product formed in (iv).

In a preferred method of the invention said oligonucleotide primer is a palindromic sequence.

A palindromic sequence is a sequence which has the same sequence when read in a 5′-3′ direction as when read in a 3′-5′ direction. For example a palindrome of the sequence: 5′ GGGCAAACGGG 3′ is 3′ GGGCAAACGGG 5′.

The use of a single palindromic primer to amplify the ssDNA allows accurate PCR conditions to be established thereby providing an reliable test.

In a preferred method of the invention said method detects two or more biological molecules.

In a further preferred method of the invention said method detects a plurality of biological agents.

An embodiment of the invention will know be described by example only and with reference to the following figures:

FIG. 1 shows a schematic diagram of an embodiment of the present invention;

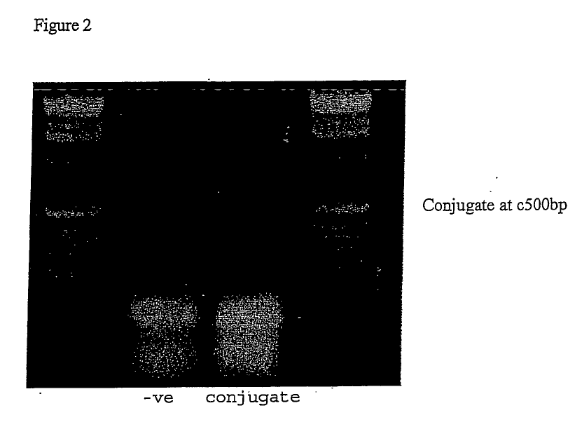

FIG. 2 is an agarose gel electrophoresis of a thyroid stimulating hormone (TSH):oligonucleotide conjugate;

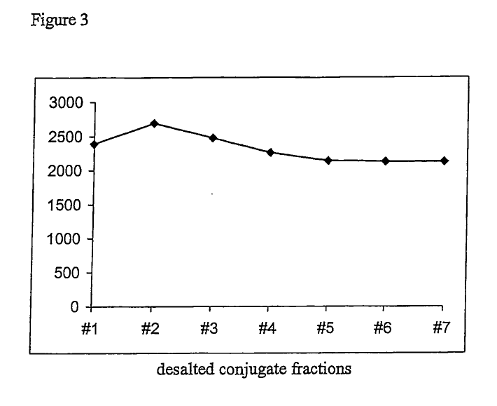

FIG. 3 is an analysis of partially purified conjugate through a PD10 column;

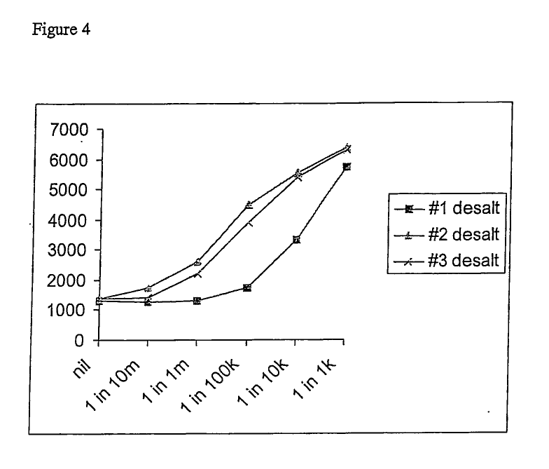

FIG. 4 is PCR amplification of conjugate aliquots from column fractions;

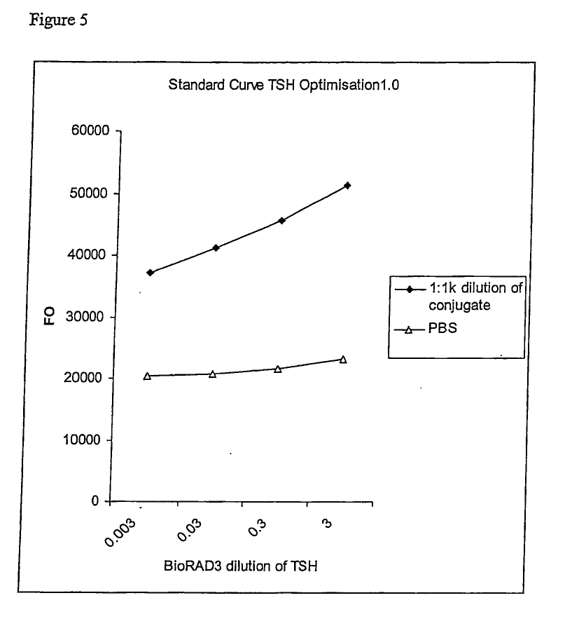

FIG. 5 is a standard curve for TSH detection using a 1:1000 dilution of conjugate; and

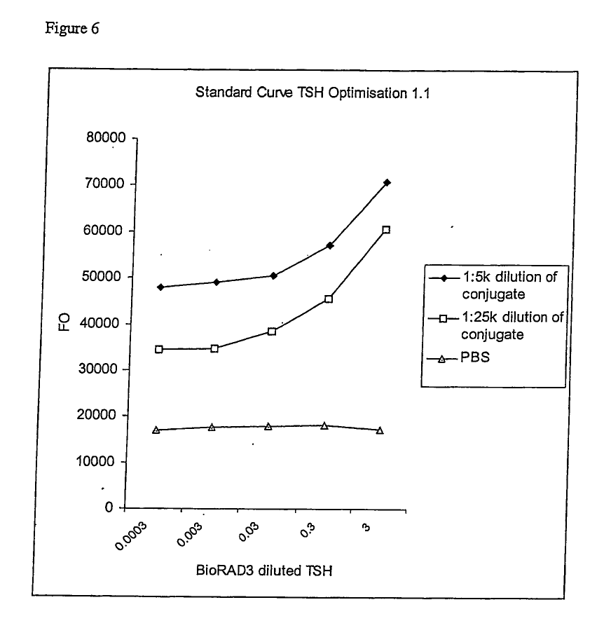

FIG. 6 is a standard curve for TSH detection using a 1:5000 and a 1:25000 dilution of conjugate;

### MATERIALS AND METHODS

### Oligonucleotide & Template Design

A 100 base single strand oligonucleotide (GAT TTA ATC TGT ATC AGG CGG GTA TGG AGT ATA ATC TAG TAG AGA GTT AAGTAT GTA ATA TCG TTA AGC TAA TCT TAT GGA TAA AAA TGC TAT GGC AT ssTemp) was designed using Oligo6 software in conjunction with database searches, together with comboC (GAT TTA ATC TGT ATC AGG CAT GCC ATA GCA TTT TTA TC) and revC (GAT TTA ATC TGT ATC AGG C) oligonucleotides. The revC oligonucleotide was designed to anneal to the 3′ terminal of the ssTemp, the comboC is designed to anneal to the 5′ terminal of the ssTemp, this oligonucleotide also contains the revC sequence at its 5′ terminal. ssTemp exhibits no significant homology with human nucleic acid sequences and contains no internal annealing sites for either comboC or revC. These oligonucleotides were commercially synthesized.

### Conjugation

500 ul of 1 mg/ml anti human TSH was desalted using a PD10 column and resuspended in 100 mM sod phos 5 mM EDTA pH6.0. Fractions containing the antibody were pooled to a volumne of 1 ml and added to 1 vial 2 mercaptoethlyamine (2-MEA) and incubated at 37′ C. for 90 mins. The solution was applied to two PD10 columns, 500 ul to each pre-equilibrated with PBS 5 mM EDTA pH7.15. 1 ml of antibody containing fractions was collected and pooled.

200 ul of amino-labeled comboC oligonucleotide 2 mg/ml in water) was added to 250 ul of water followed by 50 ul of 1M sodium phosphate pH8.0. 3 mg of succinimid 4 (N-maleimidomethyl)cyclohexane-1-carboxlate (SMCC) added, vortexed and the solution heated to 37′ C. for 20 mins. The solution was applied to a PD10 column pre-equilibrated with PBS pH7.15. Oligonucleotide containing fractions were identified using Oligreen (Molecular Probes)and pooled (1.5 ml). 1 ml of reduced anti TSH antibody and 1.5 ml SSMCC derived amino comboC oligonucleotide were pooled and concentrated to 100 ul using a Sartorius 5 Kda spin column.

Solution incubated with agitation in the dark at room temp for 2 hrs and overnight at 4′ C.1 ul of conjugate+9 ul water and 2 ul gel loading buffer (GLB) were loaded onto a 1% gel, as control 1 ul of aminocomboC was loaded in a similar manner (see FIG. 2).

### Crude Purification

25 ul of conjugate, 25 ul water and 10 ul GLB was loaded onto a 1% gel, run and the bands of 508/517 bp excised were glass wool purified and the volume, 250 ul was made up to 500 ul with PBS and applied to a PD 10 column pre-equilibrated with PBS and 7×500 ul fractions were collected. 10 ul of each fraction was diluted 1:10 and added to a goat anti-mouse coated microtitre plate and incubated for 1 hr at room temp. After washing 100 ul of 1:100 OliGreen was added and the plate read (see FIG. 3).

### PCR Amplification of Desalted Conjugate

A 16 ul aliquot of the conjugate diluted 1:1 k through to 1:10M in water was added to wells, together with 4 ul of PCR reaction mix and the PCR reaction undertaken (see FIG. 4).

Reaction Mix

10×55.0

MgCl 22.0

DNTP's 11.0

RevC 0.4

SsTemp 1.0

Water 18.0

hsTaq 2.8

Cycling

95 15:00/94 0:15 53 0:30 72 0:40 45 cycles/72 5:00

100 ul of PicoGreen was diluted in 1:200 in TE pH7.5 and added to the wells. After a 5 minute incubation the wells were read at 485/528 nm

### MAQSSE—T4 DNA Polymerase & S1 Nuclease

Forward (Fwd) and reverse (Rev) oligonucleotide primers were designed to flank the MCS of a bacterial vector. These primers were designed to consist of approximately 60% A+T and 40% G+C and exhibit no 3′ terminal dimer formation. A 750 bp insert containing no thermally significant annealing sites was cloned into the vector. Nested primers Fwdin and Revin were designed to amplify a 400 bp region within the insert, these consisted of approximately 50% A+T and 50% G+C and exhibited no 3′ terminal dimer formation. PCR was optimised in terms of Tm gradient, cycle number and reaction mix for combinations of Fwd & Rev and Fwdin & Revin.

Single-Strand PCR Template (SSPT) synthesis was achieved in a two stage process.

(i) PCR of the vector DNA was undertaken with the Fwd and Rev primers, the product was visualised by agarose gel electrophoresis, excised and purified. This was labeled QIA Fwd&Rev.

(ii) To produce a Rev primed SSPT, asymmetrical PCR was optimised in terms of QIAFwd&Rev concentration, Fwd & Rev primer ratio and cycle number. Once produced the Rev primed single strand PCR product was excised from an agarose gel and purified.

### SSPT Characterisation

To establish that Rev primed SSPT was a single strand an aliquot was diluted in a mix of 1× S1 buffer containing S1 nuclease. This was incubated for 30 minutes at 37° C. a similar reaction using QIA Fwd&Rev served as negative control. These results were evaluated by agarose gel electrophoresis.

To determine the optimal conditions for T4 DNA polymerase extension of the Fwd primer along the Rev primed SSPT, experiments were undertaken in which the serial dilutions of Rev primed SSPT were added to the T4 DNA polymerase mix and these were incubated over a range of test period. Following standard ethanol/salt precipitations and resuspension in water, PCR was undertaken using the Fwdin & Revin primers. The results were evaluated visually using agarose gel electrophoresis. Once optimal conditions were established serial dilutions of Fwd were made and the procedure repeated to demonstrate an association between diminishing Fwd primer concentrations and signal intensity.

### Fluorimetrical Quantitation

To quantify Fwd, serial dilutions were amplified using PCR after which a 1:200 dilution of PicoGREEN in TE pH 7.8 was added. The fluorophore was excited at 485 nm and the emitted light read at 528 nm these values were used to produce a standard calibration curve.

### Immunoassay 1

The platform assay used to compare the sensitivity of the technique with current labels was for human TSH (hTSH) using monoclonal antibodies from Medix Biochemica and controls/calibrators from the Department of Clinical Chemistry at the Royal Liverpool University of Hospital. Typically tracer antibody was either labeled with biotin using biotinamidocaproate NHS ester (control) or amino modified Fwd using the heterobifunctional reagent sulfo SMCC. Capture antibody was diluted in binding buffer and added to wells of 96 well polycarbonate plates and incubated overnight at room temperature. Following blocking the controls and calibrator were added to the wells and incubated overnight at room temperature. Following washing, tracer antibody was added and incubated at room temperature for four hours. In the case of the control assay, following washing, avidin D HrP was added to the wells, incubated for 30 minutes. After washing TMB was added and the reaction stopped after 30 minutes with concentrated acid. The plate was read at 450 nm. In the case of the Fwd label, following washing the T4 DNA polymerase mix including Rev primed SSPT was added to the wells and incubated at 37° C. for 45 minutes. Following washing a mix containing S1 nuclease was added and incubated at 37° C. and incubated for 30 minutes. Following a final wash stage a PCR reaction mix including the Fwdin & Revin was added and PCR undertaken. An aliquot of dilute picoGREEN was then added to each well and the plate read using the fluorometer (results not shown).

### Immunoassay 2

Wells from a 0.2 ml gamma irradiated Hybaid plate were coated with 150 ul of 2 ug/ml 5405 mab in 50 mM NaHCO3 pH8.5, ON at RT. Wells were blocked with 150 ul of 2% lactose, 0.2% BSA, 2 mM MOPS, pH7.0 for 1 hr at room temperature. 150 ul of serial diluted reconstituted BioRad3 (containing hTSH) in PBS were added to wells and incubated overnight at 4′ C. Following washing Biohit 9 washes PBS) 70 ul of dilute conjugate desalt#2 in PBS was added to the wells and incubated at 4′ C. overnight. Following washing (Biohit 9 washes PBS, 9 washes water) 10 ul of PCR reaction mix was added to the wells and the PCR reaction undertaken

### Reaction Mix

10×40.0

MgCl 16.0

dNTPs 8.0

revC 0.3

ss temp 0.8

water 332.9

hsTaq 2.0

95 15:00/94 00:15/53 00:30/72 00:40 45 cycles 72 5:00

100 ul of 1:200 PicoGreen was added and the plate read at 485/528(see FIG. 5 and FIG. 6).

### Multianalyte Methods

RevC was commercially synthesized containing a Rox fluorophore at its 5′ terminal. PCR was undertaken under the following conditions

| | ROX+ | ROX− | RevC |
|---|---|---|---|
| 10X | 10.0 | 10.0 | 10.0 |
| MgCl | 4.0 | 4.0 | 4.0 |
| dNTP's | 2.0 | 2.0 | 2.0 |
| pBADCombo 1:10 | 0.7 | 0.7 | 0.7 |
| revCROX 1:10 | 0.7 | 0.7 | revC 0.7 |
| ssTemp | 0.2 | 0.2 | 0.2 |
| water | 81.9 | 81.9 | 81.9 |
| hsTaq | 0.5 | 0.0 | 0.5 |

cycling 95 15:/ 94 :15/ 53 :30/ 72 :40 45 cycles/ 72 5:

1 ml of 1:200 dilution PicoGreen was added to each tube and the solutions mixed before transfer to cyclindrical cuvettes. Solutions were excited at 475 nm and emission was monitored in the range 500-650 nm using a spectrofluorimeter (See Rox.ppt)

10 ul of amino comboC diluted in water was added to wells followed by 10 ul of reaction mix and PCR undertaken

| | |
|---|---|
| 10X | 82.5 |
| MgCl | 33.0 |
| DNTP's | 10.0 |
| RevCrox | 0.3 |
| SsTemp | 0.75 |
| Water 283.1 | |
| HsTaq 3.0 | |

95 15:00/ 94 0:15 53 0:30 72 0:40 45cycles/ 72 5:00

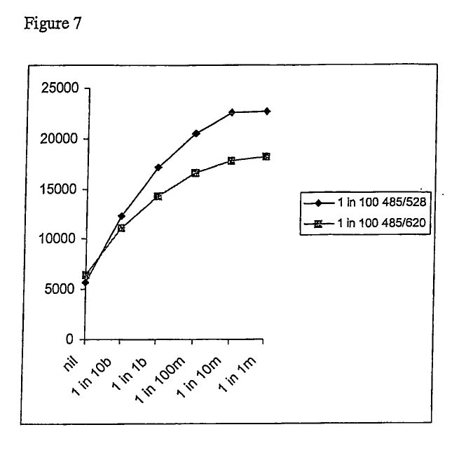

100 ul of 1:200 dilution of Picogreen in TE pH7.5 was added to the wells. After a 5 minute incubation the wells were read at 485/528 and 485/620 nm (see FIG. 7).

### MAQSSE—Palindromic

### Method

Forward (Fwd) and reverse (Rev) oligonucleotide primers were designed to flank the MCS of a bacterial vector. These primers were designed to consist of approximately 60% A+T and 40% G+C and exhibit no 3′ terminal dimer formation. The Fwd and Rev primers were synthesized together with a combined Rev+Fwd primer (Combo). A 750 bp insert containing no thermally significant annealing sites was cloned into the vector. PCR was optimised in terms of Tm gradient, cycle number and reaction mix for combinations of Fwd & Rev, Combo & Rev.

Single-Strand PCR Template (SSPT) synthesis was achieved in a two stage process.

(i) PCR of the vector DNA with the Fwd and Rev primers was undertaken, the product was visualised by agarose gel electrophoresis, excised and purified. This was labeled QIAFwd&Rev.

(ii) To produce a Rev primed SSPT, asymmetrical PCR was optimised in terms of QIAFwd&Rev concentration, Fwd & Rev primer ratio and cycle number. Once produced the Rev primed single strand PCR product was excised from an agarose gel, purified and labeled, QIAssRev.

### SSPT Characterisation

PCR was undertaken using QIAssRev and either Rev or Combo & Rev to confirm that the SSPT would bind and correctly amplify in the presence of Combo & Rev and not Rev in isolation. Serial dilutions of QIAssRev were similarly amplified and visualised by agarose gel to demonstrate an association between diminishing QIAssRev and signal intensity.

### Fluorimetrical Quantitation

To quantify QIAssRev, serial dilutions were amplified using PCR after which a 1:200 dilution of PicoGREEN in TE pH 7.8 was added. The fluorophore was excited at 485 nm and the emitted light read at 528 nm these values were used to produce a standard calibration curve.

### Comparison Immunoassay

The platform assay used to compare the sensitivity of the technique with current labels was for human TSH (hTSH) using monoclonal antibodies from Medix Biochemica and controls/calibrators from the Department of Clinical Chemistry at the Royal Liverpool University of Hospital. Typically tracer antibody was either labeled with biotin using biotinamidocaproate NHS ester (control) or amino modified Combo using the heterobifunctional reagent sulfo SMCC. Capture antibody was diluted in binding buffer and added to wells of 96 well polycarbonate plates and incubated overnight at room temperature. Following blocking the controls and calibrator were added to the wells and incubated overnight at room temperature. Following washing tracer antibody was added and incubated at room temperature for four hours. In the case of the control assay, following washing, avidin D HrP was added to the wells, incubated for 30 minutes. After washing TMB was added and the reaction stopped after 30 minutes with concentrated acid. The plate was read at 450 nm. In the case of the Combo label, following washing the PCR reaction mix was added and PCR undertaken. An aliquot of dilute picoGREEN was then added to each well and the plate read using the fluorometer.

1-23. (canceled)

24. An immuno polymerase chain reaction method of detecting at least one biological molecule in an assay sample, said method comprising:
i) providing a preparation comprising a ligand:oligonucleotide conjugate and an assay sample, wherein said ligand binds said biological molecule to be detected in said assay sample;
ii) adding a preparation comprising a single stranded template DNA molecule which anneals to said oligonucleotide in (i), deoxynucleotide triphosphates, and a DNA polymerase that elongates the primed oligonucleotide to produce a double stranded template DNA molecule; and
iii) amplifying said double stranded template DNA molecule with a polymerase chain reaction which detects said template DNA molecule using oligonucleotide primers adapted to amplify said double stranded template DNA molecule.

25. A method according to claim 24, wherein said method further comprises incubating the double stranded template DNA molecule formed in (ii) with a nuclease which degrades the single stranded template DNA.

26. A method according to claim 24, wherein said oligonucleotide primers adapted to amplify said double stranded template DNA molecule comprise a palindromic sequence.

27. A method according to claim 24, wherein said assay sample is selected from the group consisting of blood, serum, semen, lymph fluid, cerebrospinal fluid, tears, saliva, urine, and sweat.

28. A method according to claim 24, wherein said ligand is a polypeptide.

29. A method according to claim 28, wherein said polypeptide is an antibody or comprises at least the effective binding part of an antibody.

30. A method according to claim 29, wherein said antibody is a monoclonal antibody or comprises the Fab fragment of a monoclonal antibody.

31. A method according to claim 28, wherein said polypeptide is a receptor.

32. A method according to claim 28, wherein said polypeptide is a ligand of a receptor.

33. A method according to claim 32, wherein said polypeptide is thyroid stimulating hormone.

34. A method according to claim 24, wherein said biological molecule is a polypeptide and is associated with a disease condition.

35. A method according to claim 34, wherein said disease is cancer.

36. A method according to claim 35, wherein said polypeptide is a tumor rejection antigen.

37. A method according to claim 34, wherein said polypeptide is thyroid stimulating hormone.

38. A method according to claim 28, wherein said polypeptide is a polypeptide expressed by a pathogen.

39. A method according to claim 38, wherein said polypeptide is expressed by a pathogen selected from the group consisting of viruses, bacteria and parasites.

40. A method according to any of claim 24, wherein said method detects at least two biological molecules.

41. A ligand:oligonucleotide conjugate comprising at least one biological molecule bound to said ligand, which has annealed thereto a single stranded template DNA by complementary base pairing between said oligonucleotide and said template, wherein the annealed template is elongated by a DNA polymerase to form a double stranded DNA template which is subsequently amplified by a polymerase chain reaction.

42. A ligand:oligonucleotide conjugate according to claim 41, wherein said ligand is a polypeptide.

43. A ligand:oligonucleotide conjugate according to claim 42, wherein said ligand is an antibody or comprises at least the effective binding part of an antibody.

44. A conjugate according to claim 43, wherein said antibody is a monoclonal antibody.

45. A conjugate according to claim 41, wherein said biological molecule is thyroid stimulating hormone.

46. A method of detecting at least one biological molecule in a sample to be tested, said method comprising:
i) providing a preparation comprising;
a) an assay sample to be tested; and
b) a ligand:oligonucleotide conjugate;
ii) incubating said preparation under conditions which allow the binding of said conjugate to said biological molecule to form a complex;
iii) contacting the complex formed in (ii) with a single stranded DNA template nucleic acid adapted to anneal to a part of the oligonucleotide in said conjugate;
iv) providing a DNA polymerase which is capable of elongating said single stranded DNA template to form a double stranded DNA template and amplifying said double stranded DNA template in a polymerase chain reaction wherein oligonucleotide primers used in said amplification comprise a palindromic sequence.

47. A method according to claim 46, wherein said method detects a plurality of biological molecules.


SEQUENCE LISTING (5 sequences)

SEQ ID NO: 1 - DNA, 11 bp, Artificial Sequence
  oligonucleotide primer used in polymerase chain reaction
gggcaaacggg

SEQ ID NO: 2 - DNA, 11 bp, Artificial Sequence
  oligonucleotide primer used in polymerase chain reaction
gggcaaacggg

SEQ ID NO: 3 - DNA, 98 bp, Artificial Sequence
  oligonucleotide used in polymerase chain reaction
gatttaatctgtatcaggcgggtatggagtataatctagtagagagttaagtatgtaatatcgttaagctaatcttatggataaaaatgctatggcat

SEQ ID NO: 4 - DNA, 38 bp, Artificial Sequence
  oligonucleotide primer used in polymerase chain reaction
gatttaatctgtatcaggcatgccatagcatttttatc

SEQ ID NO: 5 - DNA, 19 bp, Artificial Sequence
  oligonucleotide primer used in polymerase chain reaction
gatttaatctgtatcaggc